(19) United States
(12) Patent Application Publication
(10) Pub. No.: US 2025/0072824 A1
(43) Pub. Date: Mar. 6, 2025

(54) SYSTEM AND METHOD FOR IMPROVING HYPERSOMNIA DIAGNOSTIC ACCURACY OF SLEEP ONSET DETECTION

(71) Applicant: HAPPY HEALTH, INC., Austin, TX (US)

(72) Inventors: Conrad W. Rosenbrock, Provo, UT (US); David E. Clift-Reaves, Austin, TX (US)

(21) Appl. No.: 18/822,146

(22) Filed: Aug. 31, 2024

Related U.S. Application Data
(60) Provisional application No. 63/579,992, filed on Sep. 1, 2023.

(51) Int. Cl. A61B 5/00; A61B 5/0205; A61B 5/024; A61B 5/0531; A61B 5/11

(52) U.S. Cl. CPC A61B 5/4809; A61B 5/0205; A61B 5/0531; A61B 5/1126; A61B 5/681; A61B 5/02416; A61B 5/02438; A61B 2562/0219; A61B 2562/0271

(57) ABSTRACT

A computer-implemented method for detecting cognitive stress events using a wearable electronic device is disclosed. The computer-implemented method includes (i) segmenting an EDA sensor signal into rising and falling regions; (ii) segmenting rising regions and falling regions into a plurality of rise sub-regions and a plurality of fall sub-regions, respectively; (iii) fitting a transfer function to each rise sub-region of the plurality of rise sub-regions; (iv) fitting a decaying function to each fall sub-region of the plurality of fall sub-regions; (v) combining fit parameters to identify baseline rises; (vi) combining decaying fit parameters to identify baseline falls; and (vii) identifying or characterizing physiological events, based upon the baseline rises and the baseline falls, for detecting the cognitive stress events. The wearable electronic device includes at least one electrodermal activity (EDA) sensor and at least one contact area.

### CROSS-REFERENCE TO RELATED APPLICATION

This application is a nonprovisional and claims the benefit under 35 U.S.C. § 119 (e) of U.S. Provisional Patent Application No. 63/579,992, filed Sep. 1, 2023, the contents of which are incorporated herein by reference as if fully disclosed herein.

### TECHNICAL FIELD

Embodiments described herein generally relate to a system and a method for improving hypersomnia diagnostic accuracy of sleep onset detecting using a wearable electronic device.

### BACKGROUND

Wearable electronic devices such as smart watches, fitness trackers, glucose trackers, etc., include sensors for continuous monitoring of physiological and/or biological signals of a user when a wearable electronic device is worn by the user. The sensors included in the wearable electronic device monitor health-related signals such as step count, heart rate, blood glucose level, blood pressure, respiratory rate, galvanic skin conductance (or electrodermal activity (EDA)), sleep patterns, skin temperature, and many others. Sensor data, based upon the monitored signals, helps the user to manage health status and empower the user in self-management of chronic disease conditions for diabetes, obesity, hypersomnia, and cardiovascular disease by incorporating certain lifestyle changes. However, the currently known methods of detecting sleep onsets are inaccurate to achieve diagnostic accuracy for sleep onsets that match the accuracy of physicians analyzing EEG data.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a computer-implemented method is disclosed. The computer-implemented method includes (i) performing baseline analysis of temperature sensor data; (ii) performing baseline analysis of electrodermal activity (EDA) sensor data; (iii) computing heart rate variance using Photoplethysmography (PPG) sensor data; (iv) detecting movement from accelerometry; and (v) detecting intersection of two or more regions, based upon sensor data collected from two or more different types of sensors including a thermistor, an EDA sensor, a PPG sensor, and/or an inertial measurement unit (IMU) sensor disposed at or within a body of a wearable electronic device, to detect an onset of sleep region.

In another aspect, a wearable electronic device is disclosed. The wearable electronic device includes a body, at least one memory storing instructions, at least one processor communicatively coupled with the at least one memory, and a plurality of sensors disposed at or within the body. The plurality of sensors includes one or more of a thermistor, an EDA sensor, a PPG sensor, an accelerometer, and/or an inertial measurement unit (IMU) sensor. The at least one processor is configured to execute the instructions to perform operations comprising: (i) performing baseline analysis of temperature sensor data; (ii) performing baseline analysis of electrodermal activity (EDA) sensor data; (iii) computing heart rate variance using Photoplethysmography (PPG) sensor data; (iv) detecting movement from accelerometry; and (v) detecting intersection of two or more regions, based upon sensor data collected from two or more different types of sensors of the plurality of sensors, to detect an onset of sleep region.

In yet another aspect, a wearable electronic device is disclosed. The wearable electronic device includes an annular body, at least one memory storing instructions, at least one processor communicatively coupled with the at least one memory, and a plurality of sensors disposed at or within the annular body. The plurality of sensors includes one or more of a thermistor, an EDA sensor, a PPG sensor, an accelerometer, and/or an inertial measurement unit (IMU) sensor. The at least one processor is configured to execute the instructions to perform operations comprising: (i) performing baseline analysis of temperature sensor data; (ii) performing baseline analysis of electrodermal activity (EDA) sensor data; (iii) computing heart rate variance using Photoplethysmography (PPG) sensor data; (iv) detecting movement from accelerometry; and (v) detecting intersection of two or more regions, based upon sensor data collected from two or more different types of sensors of the plurality of sensors, to detect an onset of sleep region.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A illustrates an example wearable electronic device;

FIG. 1B illustrates an example block diagram of the wearable electronic device shown in FIG. 1A; and

FIG. 2 is a flow-chart of method operations for detecting cognitive stress events using the wearable electronic device shown in FIG. 1A or FIG. 1B.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments, and, in some embodiments, it may not be included or may be combined with other features.

### DETAILED DESCRIPTION

Reference will now be made in detail to representative embodiments/aspects illustrated in the accompanying drawings. It should be understood that the following description is not intended to limit the embodiments to one preferred embodiment. On the contrary, it is intended to cover alternatives, modifications, and equivalents as can be included within the spirit and scope of the described embodiments as defined by the appended claims.

A sizable population of the world suffers from Hypersomnia. However, due to the inconvenience of attending a sleep lab for diagnostics, many continue to suffer undiagnosed. Some wearable electronic devices offer some help in this area and help a user of the wearable electronic device to detect hypersomnia. However, diagnostic accuracy of the known wearable electronic devices is questionable and generally fails to match the accuracy of diagnosis of hypersomnia by a physician using EEG data.

The present disclosure presents a system and a method for accurate hypersomnia diagnostic of sleep onset detection. The system generally includes a wearable electronic device of a small form factor, such as a ring, or a watch, that is compatible and/or comfortable to wear during sleep. Further, the wearable electronic device disclosed herein may have a small form factor.

In an example embodiment, the wearable electronic device may include one or more EDA sensors, one or more Photoplethysmography (PPG) sensors, one or more temperature sensors, and/or one or more acceleration or motion sensors. Raw sensor data from a combination of EDA, PPG, temperature, acceleration and/or motion sensors may be combined and used to achieve diagnostic accuracy substantially similar having inter-rater reliability (IRR) between physicians using EEG data. The raw sensor data from the combination of various sensors may be combined as described herein below.

By way of a non-limiting example, in an example embodiment, raw sensor data from the combination of various sensors may be combined by (i) performing baseline characterization of skin temperature to detect potential sleep onset; (ii) performing baseline characterization of EDA to find Autonomic Nervous System (ANS) evaporation events; (iii) computing Heart Rate Variability (HRV) and identifying regions of high variability; (iv) finding or identifying regions of low acceleration indicating a possible sleep state; and (v) computing a quality index based on signal durations much longer than the (individual) regions identified in the previous steps. The disclosed method intersects two or more sets of regions to find places where sleep onset may occur, as discussed below.

### Skin Temperature Baseline Characterization

In most cases, sleep onset is accompanied by a characteristic “shark fin” in the skin temperature of the individual falling asleep. In the present disclosure, a “shark fin” corresponds with a right-angled triangular waveform (with the right angle to the right) that has a much higher slope starting at the base that flattens out as it nears the top. However, shark fins may vary across multiple scales in duration in time, shape or morphology of the signal, and a range of temperature increase. Even for the same individual, the number and kind of shark fins across these multiple scales may be vastly different, depending on how the individual fall asleep. Methods for characterizing rises in an EDA signal to identify physiological events related to cognitive stress may be modified to correctly identify shark fin structures within the sensor signal measuring skin temperature. Based on the much-less-frequent sampling of the temperature sensors compared to EDA, the temperature signal may be up-sampled using interpolation. Further, even though rises are generally focus of interests, due to the riffle merging of the baseline segment identification, the parameters for falls are also important. Required individual parameter changes are also discussed in the present disclosure.

### EDA Decay Characterization

When a person falls asleep, the ANS quiets down as part of sleep, which manifests as reduced activity in the ANS, which correlates with long-term evaporation events in the EDA signal.

There is an asymmetry in the rise and fall window sizes being targeted at the different stages of the baseline rise segmentation and/or fall segmentation. Accordingly, additional baseline noise introduced into the signals may be compensated during sleep pathologies. For hypersomnia diagnosis, the time of sleep onset is critical to the diagnosis, which requires adjustment of the minimum window sizes at each step in the rise segmentation and/or fall segmentation. Rise segmentation and fall segmentation are described in detail below.

In an example embodiment, a signal is segmented into rising and falling regions. Due to noise and other environmental factors, there may be localized rises and falls that are not actual baseline changes. Accordingly, the smaller rises and falls are removed. Noise removal may be performed by a filtering technique that does not introduce a phase shift into the sensor signal. For example, the filtering technique used for noise removal may include one or more of wavelet filtering, double forward/reverse filtering, and total variance minimization. Additionally, or alternatively, any regions that are within a few seconds (or user provided time interval in seconds) of each other are merged together for signal segmentation. By way of a non-limiting example, the user provided time interval in seconds may be one second, two seconds, or five seconds, or any other time interval in seconds or milliseconds.

### Initial Rise/Fall Discovery

In an example embodiment, after the “varying” or different regions of the signal are discovered, the basic regions of rise/fall of the signal are detected using simple derivatives. If the derivatives of sufficient contiguous sets of samples are all positive, the region is constituted or identified as a rise of the sensor signal. Similarly, if the derivatives of sufficient contiguous sets of samples are all negative, the region is constituted or identified as a fall of the sensor signal.

Once the initial rise and fall regions are discovered, the discovered regions are refined to address real life environmental issues. For example, a long baseline rise is interrupted by smaller regions where the signal decreases. These smaller regions may be large enough to satisfy the criteria for minimum fall width. However, their presence interrupts the morphology of the larger rise, which is a focus of discovery of rise regions. As described herein, physiological characteristics of actual cognitive stress generally vary across scales in time, width and shape. As a result, typical pattern matching, wavelet, or other time-based feature extraction methods may not be efficient.

In an example embodiment, unnatural interruptions in rise and/or fall regions of the sensor signal are fixed by performing a series of steps in an order of jitter merging, priority unmixing, riffle merging, and variance filtering for rises. Jitter is defined as consistent oscillation between positive and negative derivatives, and jitter merging merges regions of neighboring rises or falls as long as there are no regions of the opposite kind in between them, and merging the regions will not exceed a jitter parameter threshold. In priority unmixing, two regions having overlapping edges, the region having a lower priority is discarded in favor of the region having a higher priority. When neighboring regions oscillate between rises and falls, a long rise interrupted by many smaller falls, or a long fall interrupted by many smaller rises, smaller interruptions are removed from the larger baseline in the riffle merging step. The rises that pass the jitter merging and the riffle merging, a total rise range must be large enough relative to the rest of the signal for the variance filtering step for rises; otherwise, the rises are considered as not belonging to the baseline's changes.

### Jitter Merging

Jitter is computed by counting the number of positive derivatives within a region of the sensor signal and comparing it to the number of negative derivatives. A ratio of the number of positive derivatives to the number of negative derivatives defines the jitter. When a signal is mostly increasing, with very few small deviations for signal decrease, the ratio of negative to positive derivatives will be extremely small, or the ration of positive to negative derivatives will be extremely high. Conversely, as the number of positive vs. negative derivatives reaches parity, the ratio approaches 1.

If neighboring regions with short gaps between them (for example, a few seconds) can be merged together without exceeding a jitter threshold, then such neighboring regions are combined; Otherwise, such neighboring regions are discarded. Before comparing the ratio of positive to negative derivatives, a filter is first applied so that only derivatives having absolute value larger than the threshold value are included or considered for the next step.

### Priority Unmixing

The priority unmixing step is relatively simple but necessary. As described herein, if two regions overlap, the lower priority region is discarded in favor of the higher priority region. For long rises, the rise is the higher priority region, with smaller falls having low priority region and being discarded. Similarly, for long falls, the opposite is true.

### Riffle Merging

Riffling occurs when the varying regions in the signal keep switching back and forth between rises and falls. For long changes in the baseline, the higher priority regions are kept, and the lower priority regions are merged out. For example, a long rise with small, intermittent falls may be merged into a single long rise according to a method described herein. The method described herein for the rise may also be applied for the fall. If a rise and fall are within a few seconds of each other, but the falls are shorter than a physiological threshold, then the intermittent falls are replaced with a longer, continuous rise. For long falls, if the rises are close to the falls and shorter than the physiological threshold for falls, the intermittent rises are placed with longer, continuous falls. Finally, a jitter filter is applied by removing regions whose jitter value exceeds the riffle merge threshold.

### Variance Filtering

As described herein, variance filtering is a final step for rises only, which ensures that a rise has sufficient increase in absolute signal magnitude to constitute an actual baseline shift. A tolerance is calculated relative to the standard deviation of the signal. For EDA and stress, valid baseline rises increase the signal magnitude by several times the standard deviation of the signal. Any rises below this threshold, for example, the standard deviation of the signal, are discarded.

### Gaussian Segment Analysis (Rises)

In an example embodiment, for each identified valid rise a Gaussian segment analysis is performed to find one or more sub-segments that should be fit with transfer functions, as described in detail below. The Gaussian segment analysis is performed by applying a median filter, computing the derivative of the signal and convolve a Gaussian window. Peaks and troughs in the Gaussian-convolved signal are discovered, and extrema from the signal are used as critical points dividing the signal into Gaussian segments.

### Transfer Function Fits (Rises)

In an example embodiment, once the Gaussian segments of the signal are identified, a transfer function f is fitted to each segment, for example, as y₀+Δf (α(x−x₀)). Finally, it is ensured that the objective function used for the optimization takes the derivative of the signal into account. By way of a non-limiting example, for transfer function fits of this type, the fit may have symmetric errors in certain cases so that the fit becomes a straight line with errors canceling on either side of the point where the fit and the original signal meet.

### Decaying Function Fits (Falls)

In an example embodiment, depending on the signal, one of several decaying functions may best describe the fall. The decaying function is accordingly selected that is the closest in morphology to the natural decays in the physiological baseline of the signal. By way of a non-limiting example, decaying function may include, but is not limited to, decaying exponential, power law, decaying sections of any standard statistical distribution, or linear/non-linear combinations and compositions of these functions. A non-linear fit is performed for each identified fall segment and various parameters are extracted for interpretation or characterization, as discussed herein.

### Rise Characterization

In an example embodiment, once the transfer functions have been fit to each rise segment, amplitude and slope fit parameters of the transfer function are extracted and used to create a vector describing the rise. As described herein, the physiological baseline rises in EDA for each individual will be different across scales in time, amplitude and morphology. By performing a Gaussian-based segmentation, various scaling issues may be addressed or solved. For longer periods of time, more Gaussian segments may be generated. For larger amplitude increases, larger individual segments, or more segments, may be generated depending on the morphology of the signal. Additionally, for morphological differences in scaling, a different transfer function may be selected even at the individual segment level.

By way of a non-limiting example, in an example embodiment, the amplitude and slope fit parameters from each segment are compared to the segment, and the differences between idealized fit of the segment and the segment itself are quantified for detection of cognitive stress from EDA because physiological signals tend to have the idealized shapes of transfer functions in real life. When a baseline shift is due to physiology, the signal closely represents a transfer function. When a baseline shift is due to noise, motion, or other environmental factors, the signal may approximate a transfer function somewhat, but the difference between ideal fit and baseline signal is generally greater than in the physiological case, which can be more easily detectable.

### Fall or Decay Characterization

In an example embodiment, the decaying fits are characterized by the parameters of the non-linear fit. For decays, the most important parameter may be the characteristic decay time. Whatever physiological phenomenon is being measured, it usually has time bounds associated with real, physiological responses in the body. As such, once falls are identified and had decaying functions fit to them, physiological falls are identified by their decay times falling within the region of acceptable decay times associated with the phenomenon. For cognitive stress, the ANS evaporation events happen at a microscale and a macro scale. To identify stress events, the macro scale evaporation events are the most important events occurring over a period of 5 to 20 minutes, depending on the severity of the stress event.

By combining the rise and fall characterizations, regions of actual cognitive stress may be identified that are not due to environment or noise. Further, the identified regions of actual cognitive stress may be classified as low, medium, or high stress regions using the decay constants, and the number and slope of the rise fits.

### Heart Rate Variance (HRV)

For the most part, HRV is lower during sleep in comparison to when an individual is awake. The heart generally slows as most of the body goes inactive during sleep. The exception to this is, however, rapid eye movement (REM) sleep, when brain grows more active, and the body accordingly wakes up more. HRV can be determined using one or more PPG sensors. Based upon the sensor data from one or more PPG sensors, regions likely associated with a sleep onset may be determined by performing a windowed aggregation of the HRV. One or more regions where the HRV exceeds a mean HRV by a predetermined threshold value are discovered or identified. The width of the identified region is scaled by padding with a value that is proportional to the maximum HRV. For example, if a region includes the maximum variance observed across heart rate for the entire signal, then it would pad with 30 seconds on either side of the middle of the region. In another example, a different region whose variance barely exceeded by 0.75, then standard deviations threshold would have a padding of only 5 seconds around the middle of the region. Any region whose scaled width is less than a predetermined minimum number of seconds is discarded.

### Low Acceleration Regions

During sleep, the individual generally has less movements in comparison to the movements while the individual is awake. Regions of low movements may be identified by excluding data with an acceleration magnitude greater than a first predetermined threshold (e.g., 2G), and performing a windowed aggregation of the acceleration magnitude using variance thresholding. The aggregated signal is renormalized to have the same range as the original acceleration module. Windows having magnitude less than a second predetermined threshold (e.g., 2G) are discovered or identified, which are regions of low acceleration or movements.

Additionally, or alternatively, collected raw sensor data may be discarded when at least one region of low motion or less movement is not found that has a length greater than the minimum required region size. The minimum required region size may be preconfigured or predetermined. The first predetermined threshold may be same as, or different from, the second predetermined threshold.

### Sleep Onset Detection

In an example embodiment, a sleep onset may be detected by combining multiple regions discussed herein. The inflection points, which are at the base of the front of the shark fin, in the temperature shark fins are detected. These points are at the base of the front of the shark fin. A padding in time before and after the inflection point may be added to define the temperature region. The padding time to be added before and after the inflection point may be predetermined or preconfigured. Further, the EDA decay regions are extended to their characteristic decay time (1/e such that the EDA decay region does not exceed a maximum expected decay. The EDA time is extended based upon an expected EDA decay region if not interrupted by additional stress features in the EDA signal. An intersection between an EDA decay region and a temperature shark fin region is identified, which corresponds with the first onset region. Next, an intersection between the decay region, the temperature shark fin region, and an HRV region is identified, which corresponds with a candidate for sleep onset. Additionally, or alternatively, an intersection between an HRV region and a region of low motion or less movement (also referenced herein as low acceleration) may be identified, which also corresponds with a candidate for sleep onset. Any sleep onset candidate that is not followed by a region of low acceleration within a number of seconds is discarded. The number of seconds is personal to each individual and corresponds with the duration between sleep onset and stillness for that individual. However, when there are multiple onset candidates within a preconfigured period (e.g., 8 minutes, as discussed below), a mean value computed for the onset candidates may be considered for the sleep onset.

Interrater reliability between physicians at sleep labs generally produces onset times that are only within 3 minutes of each other. A hypersomnia diagnosis requires that a subject individual fall asleep within 8 minutes of when they lie down to nap. This is the maximum constraint for the final mean onset period of 8 minutes including 4 minutes of padding before and after that is related to the 3-minute error in onset confidence among various experts.

By combining baseline analysis of temperature and EDA with heart rate variance, the regions of sleep onset are identified with accuracy rivaling physicians using EEG data. For example, using the disclosed method, accuracy of 87% may be achieved in comparison to 90% accuracy for physicians using EEG data, while mean onset error for the disclosed method is 155 seconds in comparison to 159 seconds for the physicians. Further, the disclosed method can be performed using EDA, temperature, PPG, and/or movement or motion sensors fitted in a smartwatch or a ring (a type of small form-factor wearable electronic device), hypersomnia diagnostic can be performed in a home setting with physician-level accuracy.

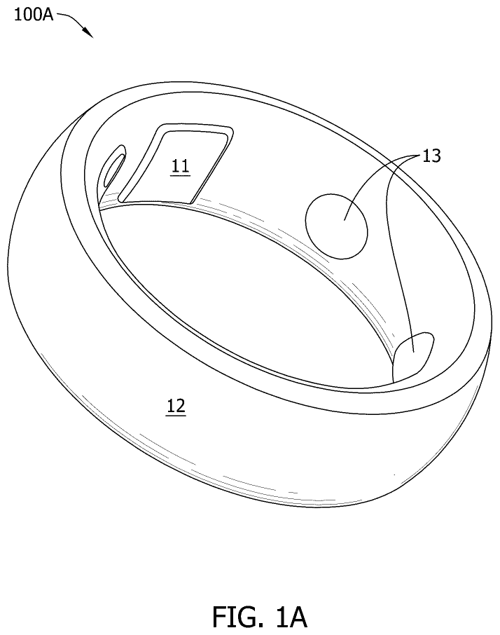

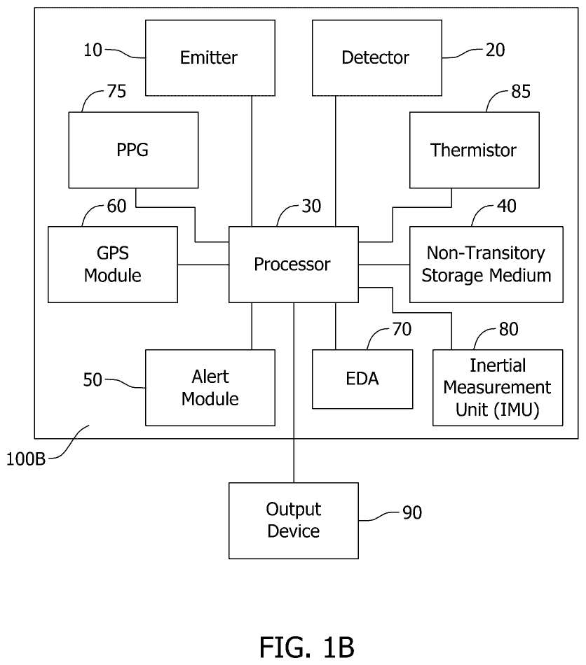

FIG. 1A illustrates an example wearable electronic device 100A. The wearable electronic device 100 may be worn by a user or secured to the user. By way of an example, the wearable electronic device may include, but is not limited to, a wearable computer, a wearable watch, a wearable communication device, a wearable media player, a wearable health monitoring device, and the like. In one example, the wearable electronic device 100A may be a wearable electronic device including multiple functionalities such as time keeping, health monitoring, sports monitoring, medical monitoring, stress monitoring, sleep monitoring, communications, navigation, computing operations, monitoring a user's physiological and/or biological signals and providing health-related information based on those signals, and/or communicating with other electronic devices or services in a wired or wireless manner. The wearable electronic device 100A may also provide alerts to the user, which may include one or more of audio, haptic, visual and/or other sensory output. The wearable electronic device 100A may also display data on a display device of the wearable electronic device 100A and acquire sensor data form one or more sensors positioned at or within the wearable electronic device 100A.

The wearable electronic device 100A includes a device body 11 including a housing that carries, encloses, and supports both externally and internally various components (including, for example, integrated circuit chips and other circuitry) to provide computing and functional operations for the wearable electronic device 100A. The components may be disposed on the outside of the housing, partially within the housing, through the housing, completely inside the housing, and the like. The housing may, for example, include a cavity for retaining components internally, holes or windows for providing access to internal components, and various features for attaching other components. The housing may also be configured to form a water-resistant or water-proof enclosure for the device body 11. For example, the housing may be formed from as a single unitary body and the openings in the unitary body may be configured to cooperate with other components to form a water-resistant or water-proof barrier. By way of a non-limiting example, the device body 11 may include components such as, but not limited to, processing units, memory, display, sensors, biosensors, speakers, microphones, haptic actuators, batteries, and so on. The wearable electronic device 100A may also include a band 12 or strap or other means for attaching to a user. In the example wearable electronic device 100A, the band 12 has an annular shape with an aperture to receive a finger of a subject user.

Additionally, the device body 11 may have one or more contact areas 13 for cognitive stress measurement for the user. By way of an example, the one or more contact areas may be provided as one or more buttons on the sides of the device body 11. Additionally, or alternatively, one or more EDA sensors, one or more temperature sensors, and/or one or more PPG sensors may be positioned on the bottom of the device body 11 such that these sensors come into contact with skin of the subject user or face the skin on the body for measuring various physiological and/or biological events.

FIG. 1B illustrates components of the wearable electronic device 100A according to an example of this disclosure. As shown in FIG. 1B, the wearable electronic device 100B includes an emitter 10 and detector 20, which are coupled to a processor 30. The processor 30 is coupled to a non-transitory storage medium 40. The wearable electronic device 100B is coupled to an output device 90. The wearable electronic device 100B is the wearable electronic device 100A shown in FIG. 1A.

The emitter 10 delivers light to a tissue and the detector 20 collects the optically attenuated signal that is backscattered from the tissue. In at least one example, the emitter 10 can be configured to emit at least three separate wavelengths of light. In another example, the emitter 10 may be configured to emit at least three separate bands or ranges of wavelengths. In at least one example, the emitter 10 may include one or more light emitting diodes (LEDs). The emitter 10 may also include a light filter. The emitter 10 may include a low-powered laser, LED, or a quasi-monochromatic light source, or any combination thereof. The emitter may emit light ranging from infrared to ultraviolet light. As indicated above, the present disclosure uses Near-Infrared Spectroscopy (NIRS) as a primary example and the other types of light can be implemented in other examples and the description as it relates to NIRS does not limit the present disclosure in any way to prevent the use of the other wavelengths of light.

The data generated by the detector 20 may be processed by the processor 30, such as a computer processor, according to instructions stored in the non-transitory storage medium 40 coupled to the processor. The processed data can be communicated to the output device 90 for storage or display to a user. The displayed processed data may be manipulated by the user using control buttons or touch screen controls on the output device 90 or on the device body 11.

The wearable electronic device 100B may include an alert module 50 configured to generate an alert. The processor 30 may send the alert to the output device 90 or the alert module 50 may send the alert directly to the output device 90. In at least one example, the wearable electronic device 100B may be configured so that the processor 30 is configured to send an alert to the output device 90 without the device including an alert module 50.

The alert may provide notice to a user, via a speaker or display on the output device 90, of a change in biological indicator conditions or other parameter being monitored by the wearable electronic device 100B, or the alert may be used to provide an updated biological indicator level to a user. In at least one example, the alert may be manifested as an auditory signal, a visual signal, a vibratory signal, or combinations thereof. In at least one example, an alert may be sent by the processor 30 when a predetermined biological indicator event occurs during a physical activity.

In at least one example, the wearable electronic device 100B may include a Global Positioning System (GPS) module 60 configured to determine geographic position and tagging the biological indicator data with location-specific information. The wearable electronic device 100B may also include an EDA sensor 70, a PPG sensor 75, an IMU 80, and a thermistor 85. The IMU 80 may be used to measure, for example, gait performance of a runner or pedal kinematics of a cyclist, as well as physiological parameters of a user during a physical activity. The EDA sensor 70, the PPG sensor 75, IMU 80, and thermistor 85 may also serve as independent sensors configured to independently measure parameters of physiological threshold. The wearable electronic device 100B may also include other types of sensors not described herein.

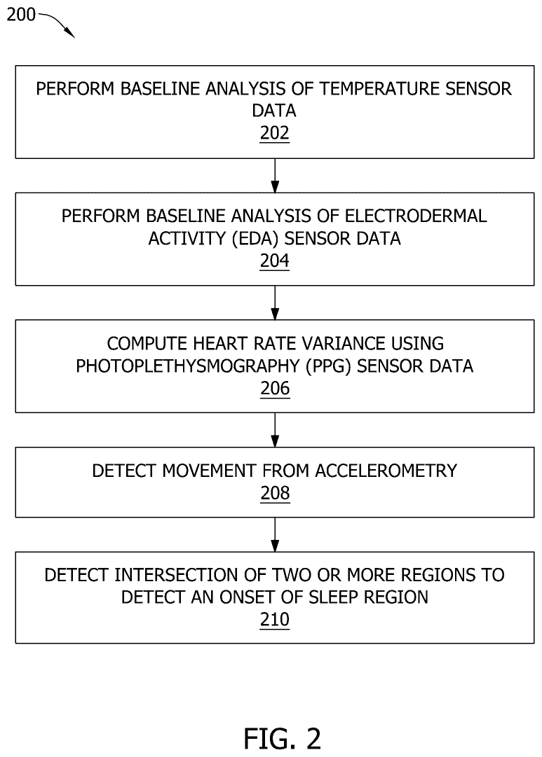

FIG. 2 is a flow-chart 200 of method operations for detecting an onset of sleep during any period time using a wearable electronic device such as the wearable electronic device 100A shown in FIG. 1A or 100B shown in FIG. 1B. The method operations include, while the wearable electronic device is on the body of the user or worn by the user in its normal operational use, performing 202 baseline analysis of temperature of a user, performing 204 baseline analysis of EDA, computing 206 HRV based upon PPG sensor signals, and detecting 208 motion of the user from accelerometry, each of which is described in detail herein, and, therefore, those details are not repeated here for the sake of brevity. The baseline analysis of temperature of the user identifies shark fin shaped signal features, and baseline analysis of EDA identifies regions of long-term ANS evaporation. As described herein, computed HRV or windowed HRV defines regions potential sleep onset, and low acceleration regions may be identified from accelerometry that is based on continuous and real-time measurement and recording of movement-induced acceleration signals. The method operations include detecting 210 intersection of two or more regions based upon sensor data collected from two or more different type of sensors including a thermistor, an EDA sensor, a PPG sensor, and/or an IMU sensor (or an accelerometer).

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B. or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed herein, that the operations and steps presented with respect to methods and techniques described herein are meant as exemplary and accordingly are not exhaustive. One may further appreciate that alternate step order or fewer or additional operations may be required or desired for particular embodiments.

Although the disclosure herein is described in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of some embodiments, whether or not such embodiments are described and whether or not such features are presented as being a part of a described embodiment. Thus, the breadth and scope of the present description should not be limited by any of the exemplary embodiments described herein but is instead defined by the claims herein presented.

What is claimed is:

1. A method comprising:
performing baseline analysis of temperature sensor data;
performing baseline analysis of electrodermal activity (EDA) sensor data;
computing heart rate variance using Photoplethysmography (PPG) sensor data;
detecting movement from accelerometry; and
detecting intersection of two or more regions, based upon sensor data collected from two or more different types of sensors including a thermistor, an EDA sensor, a PPG sensor, and/or an inertial measurement unit (IMU) sensor disposed at or within a body of a wearable electronic device, to detect an onset of sleep region.

2. The method of claim 1, wherein performing the baseline analysis of temperature sensor data includes identifying shark fin shaped signal features in the temperature sensor data.

3. The method of claim 2, wherein performing the baseline analysis of temperature sensor data further includes using transfer functions fits to segmented signals to characterize physiological shark fin shapes.

4. The method of claim 1, wherein the onset of sleep region is defined by an inflection point at a base in front of a shark fin shaped signal.

5. The method of claim 1, wherein performing the baseline analysis of EDA sensor data comprises identifying regions of long-term Autonomic Nervous System evaporation.

6. The method of claim 1, wherein the heart rate variance or a windowed aggregation of heart rate variance defines the onset of sleep region.

7. The method of claim 6, wherein a region of heart rate variance is padded by a predetermined time duration based upon a maximum heart rate variance value of the region of heart rate variance.

8. The method of claim 1, further comprising identifying a low-acceleration region or a low-motion region, based upon the detected movement from the accelerometry, using a windowed variance to identify low-motion regions.

9. The method of claim 8, wherein the detected onset of sleep region is discarded if not followed by the low-acceleration region or the low-motion region within a predetermined time duration in a few seconds, wherein the predetermined time duration is different for each subject user.

10. A wearable electronic device comprising:
a body;
a plurality of sensors disposed at or within the body, the plurality of sensors including one or more of a thermistor, an EDA sensor, a PPG sensor, an accelerometer, and/or an inertial measurement unit (IMU) sensor;
at least one memory storing instructions; and
at least one processor communicatively coupled with the at least one memory and configured to execute the instructions to perform operations comprising:
performing baseline analysis of temperature sensor data;
performing baseline analysis of EDA sensor data;
computing heart rate variance using PPG sensor data;
detecting movement from accelerometry; and
detecting intersection of two or more regions, based upon sensor data collected from two or more different types of sensors of the plurality of sensors, to detect an onset of sleep region.

11. The wearable electronic device of of claim 10, wherein performing the baseline analysis of temperature sensor data includes identifying shark fin shaped signal features in the temperature sensor data.

12. The wearable electronic device of claim 11, wherein performing the baseline analysis of temperature sensor data further includes using transfer functions fits to segmented signals to characterize physiological shark fin shapes.

13. The wearable electronic device of claim 10, wherein the onset of sleep region is defined by an inflection point at a base in front of a shark fin shaped signal.

14. The wearable electronic device of claim 10, wherein performing the baseline analysis of EDA sensor data comprises identifying regions of long-term Autonomic Nervous System evaporation.

15. The wearable electronic device of claim 10, wherein the heart rate variance or a windowed aggregation of heart rate variance defines the onset of sleep region.

16. The wearable electronic device of claim 15, wherein the operations further comprising padding a region of heart rate variance by a predetermined time duration based upon a maximum heart rate variance value of the region of heart rate variance.

17. The wearable electronic device of claim 10, wherein the operations further comprising identifying a low-acceleration region or a low-motion region, based upon the detected movement from the accelerometry, using a windowed variance to identify low-motion regions.

18. The wearable electronic device of claim 17, wherein the operations further comprising discarding the detected onset of sleep region if not followed by the low-acceleration region or the low-motion region within a predetermined time duration in a few seconds, wherein the predetermined time duration is different for each subject user.

19. The wearable electronic device of claim 10, wherein the wearable electronic device is a ring or a watch, the ring or watch has a small form factor.

20. A wearable electronic device comprising:
an annular body;
a plurality of sensors disposed at or within the annular body, the plurality of sensors including one or more of a thermistor, an EDA sensor, a PPG sensor, an accelerometer, and/or an inertial measurement unit (IMU) sensor;
at least one memory storing instructions; and
at least one processor communicatively coupled with the at least one memory and configured to execute the instructions to perform operations comprising:
performing baseline analysis of temperature sensor data;
performing baseline analysis of EDA sensor data;
computing heart rate variance using PPG sensor data;
detecting movement from accelerometry; and
detecting intersection of two or more regions, based upon sensor data collected from two or more different types of sensors of the plurality of sensors, to detect an onset of sleep region.
